(19) United States
(12) Patent Application Publication
(10) Pub. No.: US 2015/0320762 A1
(43) Pub. Date: Nov. 12, 2015

(54) TREATMENT OF PROLIFERATIVE DISEASES WITH PYRIMIDODIAZEPINONES

(71) Applicant: Cyclacel limited, London (GB)

(72) Inventors: Susan DAVIS, Angus (GB); Daniella I. ZHELEVA, Fife (GB)

(21) Appl. No.: 14/387,963

(22) PCT Filed: Mar. 28, 2013
(86) PCT No.: PCT/GB2013/050816

§ 371 Date: Sep. 25, 2014

(30) Foreign Application Priority Data
Mar. 30, 2012 (GB) 1205752.7

(51) Int. Cl. A61K 31/551; A61K 9/00

(52) U.S. Cl. CPC A61K 31/551; A61K 9/0019

(57) ABSTRACT

A first aspect of the invention relates to a compound of formula (I), or a pharmaceutically acceptable salt thereof, for use in treating a proliferative disorder, wherein: X is NR⁷; R¹ and R² are each independently H, alkyl or cycloalkyl; R³ is a 6-membered heterocycloalkyl group selected from piperidinyl, piperazinyl, morpholinyl and tetrahydropyranyl, wherein said heterocycloalkyl group is optionally further substituted by one or more (CH₂)—R¹⁹ groups; R⁴ and R^{4′} are each independently H or alkyl; or R⁴ and R^{4′} together form a spiro cycloalkyl group; Q is CH or N; R⁶ is OR⁸ or halogen; n is 1, 2 or 3; R¹⁹ is H, alkyl, aryl or a cycloalkyl group; R⁷ and R⁸ are each independently H or alkyl; and wherein said compound is administered in accordance with a dosing regimen which: (i) maintains a plasma concentration of from about 50 to about 500 nM for a period of up to about 16 hours; or (ii) maintains a plasma concentration of from about 0.5 μM to about 1 μM for a period of up to about 6 hours; or (iii) achieves a maximum plasma concentration (Cmax) of no more than about 500 nM within a period of about 6 hours; or (iv) achieves a maximum plasma concentration (Cmax) of no more than about 200 nM within a period of about 16 hours; or (v) achieves a maximum plasma concentration (Cmax) of about 0.5 μM to about 1 μM within about 6 hours. Further claims relate to a method of treatment based on this dosing regimen, and kits relating to the same.

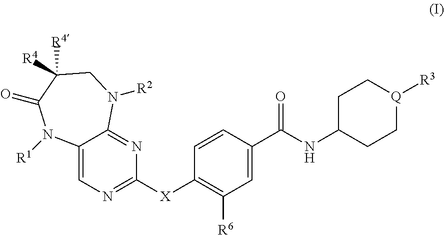

The present invention relates to a dosing regimen for treating a proliferative disorder. More specifically, but not exclusively, the invention relates to the use of small molecule Plk1 inhibitors in a particular dosing regimen that has a beneficial therapeutic window.

### BACKGROUND TO THE INVENTION

Polo-like kinases are a family of serine threonine kinases that are critical regulators of cell cycle progression and DNA damage responses (Petronczki et al. 2008). Plk1 is frequently overexpressed in cancer and its level correlates with aggressiveness and has prognostic value for predicting outcome (Kanaji et al. 2006). Cancer cell proliferation is blocked in vitro and in vivo by small Plk1 inhibitors and Plk1 antisense/siRNA (Spankuch et al. 2007). Plk1 inhibitors cause mitotic arrest and subsequent induction of apoptosis. Due to the central role of PKL1 in mitosis and cell division, rapidly proliferating normal cells are also affected by Plk1 inhibitors. As a result clinical Plk1 inhibitors have shown narrow therapeutic windows, and have been shown to cause significant haematological toxicity (Schoffski et al. 2012). Identification of patient/tumor selection markers and treatment regimens which will extend the therapeutic window is critical for the successful development of these agents. It has been shown that mutant TP53 can be one such predictive marker for sensitivity towards Plk1 inhibitors (Degenhardt et al 2011).

Previously we have identified pyrimidodiazepinone small molecules that inhibit Plk1 potently and selectively and have strong anti-proliferative activity in vitro and in vivo (Hollick et al. 2010). PLK inhibitors and their use in the treatment of proliferative disorders are described in International patent application WO 2004/067000 in the name of Cyclacel Limited.

The present invention seeks to provide a new dosing regimen for treating proliferative disorders with small molecule PLK inhibitors. More specifically, the present invention seeks to provide a dosing regimen that allows for a shorter treatment duration and an improved therapeutic window.

### STATEMENT OF INVENTION

A first aspect of the invention relates to a compound of formula (I), or a pharmaceutically acceptable salt thereof, for use in treating a proliferative disorder,

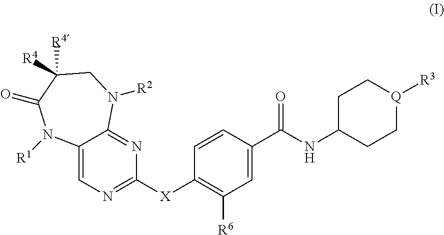

wherein:

### X is NR⁷;

R¹ and R² are each independently H, alkyl or cycloalkyl;
R³ is a 6-membered heterocycloalkyl group selected from piperidinyl, piperazinyl, morpholinyl and tetrahydropyranyl, wherein said heterocycloalkyl group is optionally further substituted by one or more (CH₂)ₙR¹⁹ groups;
R⁴ and R^{4′} are each independently H or alkyl; or
R⁴ and R^{4′} together form a spiro cycloalkyl group;

### Q is CH or N;

R⁶ is OR⁸or halogen;
n is 1, 2 or 3;
R¹⁹ is H, alkyl, aryl or a cycloalkyl group;
R⁷ and R⁸ are each independently H or alkyl; and
wherein said compound is administered in accordance with a dosing regimen which:
(i) maintains a plasma concentration of from about 50 nM to about 500 nM for a period of up to about 16 hours; or
(ii) maintains a plasma concentration of from about 0.5 μM to about 1 μM for a period of up to about 6 hours; or
(iii) achieves a maximum plasma concentration (Cmax) of no more than about 500 nM within a period of about 6 hours; or
(iv) achieves a maximum plasma concentration (Cmax) of no more than about 200 nM within a period of about 16 hours; or
(v) achieves a maximum plasma concentration (Cmax) of about 0.5 μM to about 1 μM within about 6 hours.

A second aspect of the invention relates to a method of treating a proliferative disorder, said method comprising administering to a subject a therapeutically effective amount of a compound of formula (I), or a pharmaceutically acceptable salt thereof, as defined above, in accordance with a dosing regimen which:
(i) maintains a plasma concentration of from about 50 nM to about 500 nM for a period of up to about 16 hours; or
(ii) maintains a plasma concentration of from about 0.5 μM to about 1 μM for a period of up to about 6 hours; or
(iii) achieves a maximum plasma concentration (Cmax) of no more than about 500 nM within a period of about 6 hours; or
(iv) achieves a maximum plasma concentration (Cmax) of no more than about 200 nM within a period of about 16 hours; or
(v) achieves a maximum plasma concentration (Cmax) of about 0.5 μM to about 1 μM within about 6 hours.

A third aspect of the invention relates to the use of a compound of formula (I), or a pharmaceutically acceptable salt thereof, as defined above, in the preparation of a medicament for treating a proliferative disorder, wherein said compound is administered in accordance with dosing regimen which:
(i) maintains a plasma concentration of from about 50 nM to about 500 nM for a period of up to about 16 hours; or
(ii) maintains a plasma concentration of from about 0.5 μM to about 1 μM for a period of up to about 6 hours; or
(iii) achieves a maximum plasma concentration (Cmax) of no more than about 500 nM within a period of about 6 hours; or
(iv) achieves a maximum plasma concentration (Cmax) of no more than about 200 nM within a period of about 16 hours; or
(v) achieves a maximum plasma concentration (Cmax) of about 0.5 μM to about 1 μM within about 6 hours.

A fourth aspect of the invention relates to a kit comprising:
(a) a compound of formula (I), or a pharmaceutically acceptable salt thereof, as defined above;
(b) optionally at least one pharmaceutically acceptable diluent, excipient or carrier; and
(c) instructions to administer the compound of formula (I) in accordance with a dosing regimen which:
   (i) maintains a plasma concentration of from about 50 nM to about 500 nM for a period of up to about 16 hours; or
   (ii) maintains a plasma concentration of from about 0.5 μM to about 1 μM fora period of up to about 6 hours; or
   (iii) achieves a maximum plasma concentration (Cmax) of no more than about 500 nM within a period of about 6 hours; or
   (iv) achieves a maximum plasma concentration (Cmax) of no more than about 200 nM within a period of about 16 hours; or
   (v) achieves a maximum plasma concentration (Cmax) of about 0.5 μM to about 1 μM within about 6 hours.

A fifth aspect of the invention relates to a compound of formula (I), or a pharmaceutically acceptable salt thereof, as defined above for use in treating a proliferative disorder, wherein said compound is administered by intravenous infusion for about 1 to about 4 hours at a rate of about 0.02 to about 0.08 mg/kg/minute.

A sixth aspect of the invention relates to a compound of formula (I), or a pharmaceutically acceptable salt thereof, as defined above for use in treating a proliferative disorder, wherein said compound is administered by intravenous infusion for about 1 to about 10 hours at a rate of about 0.01 to about 0.04 mg/kg/minute.

### DETAILED DESCRIPTION

As mentioned above, a first aspect of the invention relates to a compound of formula (I), or a pharmaceutically acceptable salt thereof, for use in treating a proliferative disorder, wherein said compound is administered in accordance with a dosing regimen which:
(i) maintains a plasma concentration of from about 50 nM to about 500 nM for a period of up to about 16 hours; or
(ii) maintains a plasma concentration of from about 0.5 μM to about 1 μM for a period of up to about 6 hours; or
(iii) achieves a maximum plasma concentration (Cmax) of no more than about 500 nM within a period of about 6 hours; or
(iv) achieves a maximum plasma concentration (Cmax) of no more than about 200 nM within a period of about 16 hours; or
(v) achieves a maximum plasma concentration (Cmax) of about 0.5 μM to about 1 μM within about 6 hours.

Clinical Plk1 inhibitors investigated to date have demonstrated very narrow therapeutic windows. Most commonly, dose limiting toxicities and adverse events for these agents are grade 3/4 neutropenia and febrile neutropenia, thrombocytopenia and anemia, which are related to the inhibition of rapidly proliferating blood cells. Advantageously, the presently claimed dosing regimen provides a highly controlled, short term treatment.

In this regard, PK studies have demonstrated that pyrimidodiazepinone Plk1 inhibitors of formula (I) have a significantly shorter half-life in humans than clinical PLK inhibitors investigated to date. By way of example, MK1496 has a half-life of 25-47 hours (Doi et al., 2011), GSK461364 has a half-life of 9-13 hours (Olmos et al. 2011), BI2536 has a half-life of greater than 25 hours (Gandhi et al., 2009), and BI6727 has a half-life of about 110 hours (Gil et al., 2010). In contrast, the compounds of formula (I) typically have a much shorter half-life, which allows for shorter treatment times and an improved therapeutic window.

Moreover, the presently claimed dosing regimen also allows differentiation between sensitive cancer cells and normal proliferating cells, thereby achieving a better therapeutic window. Surprisingly, using the compounds of formula (I) the applicant has discovered that very short treatment periods, such as about 6 hours, have a much lower anti-proliferative effect on non-cancerous proliferating cells while retaining a strong anti-proliferative effect on cancerous cells.

### Plk1 Inhibitors

Compounds suitable for use in the invention are defined by formula (I) above.

As used herein, the term “alkyl” includes both saturated straight chain and branched alkyl groups which may be substituted (mono- or poly-) or unsubstituted. Preferably, the alkyl group is a C₁₋₂₀ alkyl group, more preferably a C₁₋₁₅, more preferably still a C₁₋₁₂ alkyl group, more preferably still, a C₁₋₆ alkyl group, more preferably a C₁₋₃ alkyl group. Particularly preferred alkyl groups include, for example, methyl, ethyl, propyl, isopropyl, butyl, isobutyl, tert-butyl, pentyl and hexyl. Suitable substituents include, for example, one or more halo, alkoxy, nitro, CN, NH₂, NH(alkyl) or N(alkyl)₂ groups. Preferably, the alkyl group is unsubstituted.

As used herein, the term “cycloalkyl” refers to a cyclic alkyl group which may be substituted (mono- or poly-) or unsubstituted. Preferably, the cycloalkyl group is a C₃₋₁₂ cycloalkyl group, more preferably a C₃₋₆ cycloalkyl group. Suitable substituents include, for example, one or more halo, alkoxy, nitro, CN, NH₂, NH(alkyl) or N(alkyl)₂ groups.

As used herein, the term “aryl” refers to a C₆₋₁₂ aromatic group which may be substituted (mono- or poly-) or unsubstituted. Typical examples include phenyl and naphthyl etc. Suitable substituents include, for example, one or more halo, alkoxy, nitro, CN, NH₂, NH(alkyl) or N(alkyl)₂ groups.

As used herein, the term “heterocycloalkyl” refers to a cyclic aliphatic group which contains one or more heteroatoms. Preferred heterocycloalkyl groups include piperidinyl, pyrrolidinyl, piperazinyl, tetrahydropyranyl, thiomorpholinyl and morpholinyl. More preferably, the heterocycloalkyl group is selected from N-piperidinyl, N-pyrrolidinyl, N-piperazinyl, tetrahydropyranyl and N-morpholinyl.

In one preferred embodiment, the compound is of formula (Ia), or a pharmaceutically acceptable salt thereof,

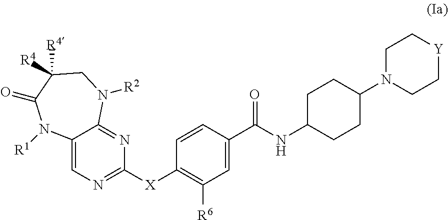

wherein Y is O or N—(CH₂)ₙR¹⁹; and
X, R¹, R², R⁴, R^{4′}, R⁶, R⁷, R⁸, n and R¹⁹ are as defined in claim 1.

In one preferred embodiment, the compound is of formula (Ib), or a pharmaceutically acceptable salt thereof,

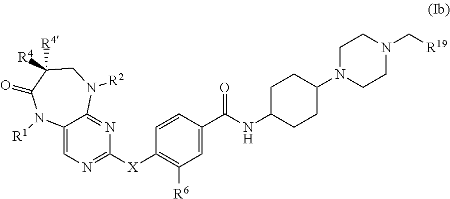

wherein X, R¹, R², R⁴, R^{4′}, R⁶, R⁷, R⁸ and R¹⁹ are as defined in claim 1.

In one preferred embodiment of the invention, R¹ is H or alkyl, more preferably, methyl.

In one preferred embodiment of the invention, R² is cycloalkyl, more preferably, cyclopentyl or cyclohexyl.

In one preferred embodiment of the invention, R⁷ is H or alkyl, more preferably, H.

In one preferred embodiment of the invention, R⁴ and R^{4′} are each independently alkyl, more preferably, methyl.

In one preferred embodiment of the invention, R⁴ and R^{4′} together form a spiro cycloalkyl group, more preferably, a spiro C₃ cycloalkyl group.

In one preferred embodiment of the invention, R⁶ is OR⁸, more preferably, OMe.

In one preferred embodiment of the invention, R¹⁹ is cyclopropyl.

In one particularly preferred embodiment the compound for use in accordance with the invention is selected from the following:

| | Name |
|---|---|
| A1 | 4-(9′-cyclopentyl-5′-methyl-6′-oxo-5′,6′,8′,9′-tetrahydrospiro[cyclopropane-1,7′- |
| | pyrimido[4,5-b][1,4]diazepine]-2′-ylamino)-N-((trans)-4-(4- |
| | (cyclopropylmethyl)piperazin-1-yl)cyclohexyl)-3-methoxybenzamide |
| A2 | 4-(9-cyclopentyl-5,7,7-trimethyl-6-oxo-6,7,8,9-tetrahydro-5H-pyrimido[4,5- |
| | b][1,4]diazepin-2-ylamino)-N-((trans)-4-(4-(cyclopropylmethyl)piperazin-1- |
| | yl)cyclohexyl)-3-methoxybenzamide |
| A3 | 4-(9′-cyclopentyl-5′-methyl-6′-oxo-5′,6′,8′,9′-tetrahydrospiro[cyclobutane-1,7′- |
| | pyrimido[4,5-b][1,4]diazepine]-2′-ylamino)-N-((trans)-4-(4- |
| | (cyclopropylmethyl)piperazin-1-yl)cyclohexyl)-3-methoxybenzamide |
| A4 | 4-(9-cyclopentyl-5-methyl-6-oxo-6,7,8,9-tetrahydro-5H-pyrimido[4,5- |
| | b][1,4]diazepin-2-ylamino)-N-((trans)-4-(4-(cyclopropylmethyl)piperazin-1- |
| | yl)cyclohexyl)-3-methoxybenzamide |
| A5 | 4-(9′-cyclopentyl-5′-methyl-6′-oxo-5′,6′,8′,9′-tetrahydrospiro[cyclopropane-1,7′- |
| | pyrimido[4,5-b][1,4]diazepine]-2′-ylamino)-N-((trans)-4-(4-ethylpiperazin-1- |
| | yl)cyclohexyl)-3-methoxybenzamide |
| A6 | 4-(9′-cyclopentyl-5′-methyl-6′-oxo-5′,6′,8′,9′-tetrahydrospiro[cyclopropane-1,7′- |
| | pyrimido[4,5-b][1,4]diazepine]-2′-ylamino)-N-((cis)-4-(4-ethylpiperazin-1- |
| | yl)cyclohexyl)-3-methoxybenzamide |
| A7 | 4-(9′-cyclopentyl-5′-methyl-6′-oxo-5′,6′,8′,9′-tetrahydrospiro[cyclopropane-1,7′- |
| | pyrimido[4,5-b][1,4]diazepine]-2′-ylamino)-N-((trans)-4-(4-methylpiperazin-1- |
| | yl)cyclohexyl)-3-methoxybenzamide |
| A8 | 4-(9′-cyclopentyl-5′-methyl-6′-oxo-5′,6′,8′,9′-tetrahydrospiro[cyclopropane-1,7′- |
| | pyrimido[4,5-b][1,4]diazepine]-2′-ylamino)-N-((cis)-4-(4-methylpiperazin-1- |
| | yl)cyclohexyl)-3-methoxybenzamide |
| A9 | N-((trans)-4-(4-benzylpiperazin-1-yl)cyclohexyl)-4-(9′-cyclopentyl-5′-methyl-6′- |
| | oxo-5′,6′,8′,9′-tetrahydrospiro[cyclopropane-1,7′-pyrimido[4,5- |
| | b][1,4]diazepine]-2′-ylamino)-3-methoxybenzamide |
| A10 | N-((cis)-4-(4-benzylpiperazin-1-yl)cyclohexyl)-4-(9′-cyclopentyl-5′-methyl-6′- |
| | oxo-5′,6′,8′,9′-tetrahydrospiro[cyclopropane-1,7′-pyrimido[4,5- |
| | b][1,4]diazepine]-2′-ylamino)-3-methoxybenzamide |
| A11 | 4-(9-cyclopentyl-5,7,7-trimethyl-6-oxo-6,7,8,9-tetrahydro-5H-pyrimido[4,5- |
| | b][1,4]diazepin-2-ylamino)-3-methoxy-N-((trans)-4- |
| | morpholinocyclohexyl)benzamide |
| A12 | 4-(9-cyclopentyl-5,7,7-trimethyl-6-oxo-6,7,8,9-tetrahydro-5H-pyrimido[4,5- |
| | b][1,4]diazepin-2-ylamino)-3-methoxy-N-((cis)-4- |
| | morpholinocyclohexyl)benzamide |
| A13 | 4-(9-cyclopentyl-5-methyl-6-oxo-6,7,8,9-tetrahydro-5H-pyrimido[4,5- |
| | b][1,4]diazepin-2-ylamino)-3-methoxy-N-[1-(tetrahydro-pyran-4-yl)-piperidin-4- |
| | yl]-benzamide |
| A14 | 4-(9′-cyclopentyl-5′-methyl-6′-oxo-5′,6′,8′,9′-tetrahydrospiro[cyclopropane-1,7′- |
| | pyrimido[4,5-b][1,4]diazepine]-2′-ylamino)-3-methoxy-N-((trans)-4- |
| | morpholinocyclohexyl)benzamide |
| A15 | 4-(9′-cyclopentyl-5′-methyl-6′-oxo-5′,6′,8′,9′-tetrahydrospiro[cyclopropane-1,7′- |
| | pyrimido[4,5-b][1,4]diazepine]-2′-ylamino)-3-methoxy-N-((cis)-4- |
| | morpholinocyclohexyl)benzamide |

and pharmaceutically acceptable salts thereof.

The compounds for use in the invention are capable of inhibiting Plk1. In one particularly preferred embodiment, the compound exhibits an IC₅₀ value for kinase inhibition of less than about 20 μM, more preferably less than about 10 μM, more preferably less than about 5 μM, more preferably still less than about 1 μM or about 0.5 μM, more preferably less than about 0.1 μM, even more preferably, less than about 0.01 μM. In one particularly preferred embodiment, the compound is selected from A1, A7 and A13.

### Dosing Regimen

As used herein, the term “plasma concentration” refers to the plasma concentration of the compound of formula (I) as measured by any suitable technique, for example, such as standard LC-MS/MS analysis. Further details of this technique are described in the accompanying examples section.

In one preferred embodiment, the compound is administered in a dosing regimen which maintains a plasma concentration of from about 100 to about 500 nM for a period of up to about 16 hours, preferably up to about 14 hours, more preferably up to about 12 hours, more preferably up to about 10 hours, more preferably up to about 8 hours, more preferably up to about 6 hours.

In one preferred embodiment, the compound is administered in a dosing regimen which maintains a plasma concentration of from about 100 to about 500 nM for a period of up to about 6 hours.

In one preferred embodiment, the compound is administered in a dosing regimen which maintains a plasma concentration of from about 100 to about 500 nM for a period of from about 3 to about 6 hours, more preferably about 4 to 6 hours, even more preferably about 5 to about 6 hours.

The time periods referred to herein are defined as the time during which the plasma concentration of the compound of formula (I) is maintained above a certain concentration, or within a certain concentration range, i.e. the period measured from the time at which the plasma concentration first reaches the required level to the time at which the plasma concentration drops below the required level.

In one preferred embodiment, the compound is administered in a dosing regimen which maintains a plasma concentration of from about 50 to about 250 nM, more preferably from about 50 to about 200 nM, for a period of up to about 16 hours.

In one preferred embodiment, the compound is administered in a dosing regimen which maintains a plasma concentration of from about 50 to about 250 nM, more preferably from about 50 to about 200 nM, for a period of about 10 to about 16 hours, more preferably about 12 to 16 hours, even more preferably about 14 to about 16 hours.

In one preferred embodiment, the compound is administered in a dosing regimen which maintains a plasma concentration of from about 0.5 μM to about 1 μM for a period of from about 3 to about 6 hours.

In one preferred embodiment, the compound is administered in a dosing regimen which achieves a maximum plasma concentration (Cmax) of about 0.5 μM to about 1 μM within about 6 hours, more preferably within about 3 to about 6 hours, more preferably within about 4 to about 6 hours, even more preferably within about 5 to about 6 hours.

As used herein, the term “Cmax” refers to the time taken for the compound of formula (I) to reach its maximum or peak plasma concentration as measured from the time of administration of the drug.

In one preferred embodiment, the compound is administered in a dosing regimen which achieves a maximum plasma concentration (Cmax) of about 100 nM to about 500 nM within a period of about 6 hours, more preferably within about 3 to about 6 hours, more preferably within about 4 to about 6 hours, even more preferably about 5 to about 6 hours.

In one preferred embodiment, the said compound is administered in a dosing regimen which achieves a maximum plasma concentration (Cmax) of about 50 nM to about 200 nM within a period of about 16 hours, more preferably within about 10 to about 16 hours, more preferably within about 12 to about 16 hours, even more preferably within about 14 to about 16 hours.

In one preferred embodiment, the compound is administered by intravenous infusion.

In one preferred embodiment, the compound is administered by intravenous infusion for about 1 to about 4 hours at a rate of about 0.04 to about 0.08 mg/kg/minute.

In one preferred embodiment, the compound is administered by intravenous infusion for about 1 to about 2 hours at a rate of about 0.02 to about 0.06 mg/kg/minute.

In one preferred embodiment, the compound is administered by intravenous infusion for about 1 to about 2 hours at a rate of about 0.02 to about 0.08 mg/kg/minute.

In one preferred embodiment, the compound is administered by intravenous infusion for about 1 to about 10 hours at a rate of about 0.01 to about 0.04 mg/kg/minute.

In one preferred embodiment, the compound of formula (I) is administered by intravenous infusion for about 1 to about 10 hours, more preferably, 1 to 8 hours, at a rate of about 0.02 to about 0.04 mg/kg/minute.

In one preferred embodiment, the compound of formula (I) is compound A13 and is administered by intravenous infusion for about 1 to about 4 hours at a rate of about 0.04 to about 0.08 mg/kg/minute.

In one preferred embodiment, the compound of formula (I) is compound A1 and is administered by intravenous infusion for about 1 to about 2 hours at a rate of about 0.02 to about 0.06 mg/kg/minute.

In one preferred embodiment, the compound of formula (I) is compound A7 and is administered by intravenous infusion for about 1 to about 3 hours at a rate of about 0.02 to about 0.08 mg/kg/minute.

In one preferred embodiment, the compound of formula (I) is compound A7 and is administered by intravenous infusion for about 1 to about 10 hours at a rate of about 0.01 to about 0.04 mg/kg/minute.

In one preferred embodiment, the compound is administered by bolus injection. The injection may be administered by intravenous, intramuscular, intrathecal or subcutaneous injection.

In one preferred embodiment, the compound is administered by two or more bolus injections. For example, the compound of formula (I) may be administered in two or more separate injections (e.g. two, three or four injections) separated by appropriate time intervals and in amounts suitable for achieving the desired plasma concentrations or Cmax values. The skilled person would be able to determine the number, timing and appropriate amounts of compound to achieve the desired plasma concentrations or Cmax values based on their common general knowledge.

### Therapeutic Use

The compounds of formula (I) have been found to possess anti-proliferative activity and are therefore believed to be of use in the treatment of proliferative disorders such as cancers, leukaemias and other disorders associated with uncontrolled cellular proliferation such as psoriasis and restenosis.

As used herein the phrase “preparation of a medicament” includes the use of one or more of the above described compounds directly as the medicament in addition to its use in a screening programme for further antiproliferative agents or in any stage of the manufacture of such a medicament.

As defined herein, an anti-proliferative effect within the scope of the present invention may be demonstrated by the ability to inhibit cell proliferation in an in vitro whole cell assay, or on the basis of kinase inhibitory activity, wherein the kinase in question is known to be linked to a particular proliferative disorder. Using such assays it may be determined whether a compound is anti-proliferative in the context of the present invention.

Preferably, the proliferative disorder is a cancer or leukaemia. The term proliferative disorder is used herein in a broad sense to include any disorder that requires control of the cell cycle, for example cardiovascular disorders such as restenosis and cardiomyopathy, auto-immune disorders such as glomerulonephritis and rheumatoid arthritis, dermatological disorders such as psoriasis, anti-inflammatory, anti-fungal, antiparasitic disorders such as malaria, emphysema and alopecia. In these disorders, the compounds of the present invention may induce apoptosis or maintain stasis within the desired cells as required.

The compounds of the invention may inhibit any of the steps or stages in the cell cycle, for example, formation of the nuclear envelope, exit from the quiescent phase of the cell cycle (G0), G1 progression, chromosome decondensation, nuclear envelope breakdown, START, initiation of DNA replication, progression of DNA replication, termination of DNA replication, centrosome duplication, G2 progression, activation of mitotic or meiotic functions, chromosome condensation, centrosome separation, microtubule nucleation, spindle formation and function, interactions with microtubule motor proteins, chromatid separation and segregation, inactivation of mitotic functions, formation of contractile ring, and cytokinesis functions. In particular, the compounds of the invention may influence certain gene functions such as chromatin binding, formation of replication complexes, replication licensing, phosphorylation or other secondary modification activity, proteolytic degradation, microtubule binding, actin binding, septin binding, microtubule organising centre nucleation activity and binding to components of cell cycle signalling pathways.

In one preferred embodiment of the invention, the proliferative disorder is cancer or leukaemia, more preferably cancer.

In one preferred embodiment, the proliferative disorder is a solid tumour.

In another preferred embodiment, the proliferative disorder is a hematological cancer. Preferably, the haematological cancer is leukaemia, more preferably, advanced leukemias or myelodysplastic syndromes (MDS). Other examples include acute myelogenous leukemia (AML), acute lymphocytic leukemia (ALL) or chronic lymphocytic leukemia (CLL).

In one preferred embodiment of the invention, the cancer comprises a non-functional p53 protein. As used herein, the term “non-functional p53 protein” includes cancers in which p53 is inactive, deficient, lacking (p53 null), absent, mutant, or not present in a functional amount.

In one preferred embodiment of the invention, the cancer comprises a p53-mutation. As used herein, the term “p53-mutation” refers to cancers comprising a mutation in the p53 protein, or in the TP3 gene encoding the p53 protein.

In one particularly preferred embodiment of the invention, the cancer is selected from breast cancer, colorectal cancer, prostate cancer, oesophageal cancer and lung cancer, more preferably, oesophageal cancer.

In one highly preferred embodiment, the cancer is oesophageal cancer.

### Pharmaceutical Compositions

Even though the compounds of formula (I) (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

### Salts/Esters

The compounds of formula (I) can be present as salts or esters, in particular pharmaceutically acceptable salts or esters.

Pharmaceutically acceptable salts of the compounds of formula (I) include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Preferably, the salt is an HCl salt.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

### Enantiomers/Tautomers

In all aspects of the present invention previously discussed, the invention includes, where appropriate the use of all enantiomers and tautomers of the compounds of formula (I). The person skilled in the art will recognise compounds that possess an optical properties (one or more chiral carbon atoms) or tautomeric characteristics. The corresponding enantiomers and/or tautomers may be isolated/prepared by methods known in the art.

### Stereo and Geometric Isomers

Some of the compounds of formula (I) may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those inhibitor agents, and mixtures thereof. The terms used in the claims encompass these forms, provided said forms retain the appropriate functional activity (though not necessarily to the same degree).

The present invention also includes the use of all suitable isotopic variations of the agent or pharmaceutically acceptable salt thereofs. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

### Solvates

The present invention also includes the use of solvate forms of the compounds of formula (I). The terms used in the claims encompass these forms.

### Polymorphs

The invention furthermore relates to the use of compounds of formula (I) in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

### Prodrugs

The invention further includes the use of compounds of formula (I) in prodrug form. Such prodrugs are generally compounds of the invention wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

### Administration

The pharmaceutical compositions for use in the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 250 mg and more preferably from 10-100 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

In one highly preferred embodiment of the invention, the compound is administered by intravenous infusion.

In another highly preferred embodiment of the invention, the compound is administered by one or more bolus injections.

### Dosage

A person of ordinary skill in the art can easily determine without undue experimentation an appropriate dose of one of the instant compositions to administer to a subject in order to achieve the required plasma concentrations and/or Cmax values. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.01 to 30 mg/kg body weight, such as from 0.1 to 10 mg/kg, more preferably from 0.1 to 1 mg/kg body weight.

Depending upon the need, the agent may be administered at a dose of from 0.01 to 48 mg/kg body weight, such as from 5 to 50 mg/kg, 0.5 to 5 mg/kg, more preferably from 2.0 to 20 mg/kg body weight.

In an exemplary embodiment, one or more doses of 10 to 150 mg/day will be administered to the patient for the treatment of malignancy.

In an exemplary embodiment, one or more doses of 50 to 1500 mg/day will be administered to the patient for the treatment of malignancy.

### Combinations

In a particularly preferred embodiment, the one or more compounds of the invention are for use in combination with one or more other active agents, for example, existing anticancer drugs available on the market. In such cases, the compounds of the invention may be administered consecutively, simultaneously or sequentially with the one or more other active agents.

Anticancer drugs in general are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining chemotherapeutic drugs are that it may promote additive or possible synergistic effects through biochemical interactions and also may decrease the emergence of resistance in early tumor cells which would have been otherwise responsive to initial chemotherapy with a single agent. An example of the use of biochemical interactions in selecting drug combinations is demonstrated by the administration of leucovorin to increase the binding of an active intracellular metabolite of 5-fluorouracil to its target, thymidylate synthase, thus increasing its cytotoxic effects. Numerous combinations are used in current treatments of cancer and leukemia. A more extensive review of medical practices may be found in “Oncologic Therapies” edited by E. E. Vokes and H. M. Golomb, published by Springer.

Beneficial combinations may be suggested by studying the growth inhibitory activity of the test compounds with agents known or suspected of being valuable in the treatment of a particular cancer initially or cell lines derived from that cancer. This procedure can also be used to determine the order of administration of the agents, i.e. before, simultaneously, or after delivery. Such scheduling may be a feature of all the cycle acting agents identified herein.

The present invention is further illustrated by way of the following non-limiting examples.

### EXAMPLES

### Compound Preparation

Compounds of formula (I), such as Examples A1 to A15 described herein and shown below, were prepared in accordance with the methods described in WO 2009040566 (Cyclacel Limited).

| | Name | |
|---|---|---|
| A1 | 4-(9′-cyclopentyl-5′-methyl-6′-oxo-5′,6′,8′,9′- tetrahydrospiro[cyclopropane-1,7′- pyrimido[4,5-b][1,4]diazepine]-2′-ylamino)-N- ((trans)-4-(4-(cyclopropylmethyl)piperazin-1- yl)cyclohexyl)-3-methoxybenzamide | 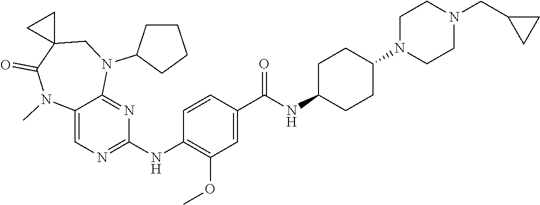 [SMILES: COc1cc(C(=O)N[C@H]2CC[C@H](N3CCN(CC4CC4)CC3)CC2)ccc1Nc1ncc2c(n1)N(C1CCCC1)CC1(CC1)C(=O)N2C] |
| A2 | 4-(9-cyclopentyl-5,7,7-trimethyl-6-oxo- 6,7,8,9-tetrahydro-5H-pyrimido[4,5- b][1,4]diazepin-2-ylamino)-N-((trans)-4-(4- (cyclopropylmethyl)piperazin-1- yl)cyclohexyl)-3-methoxybenzamide | 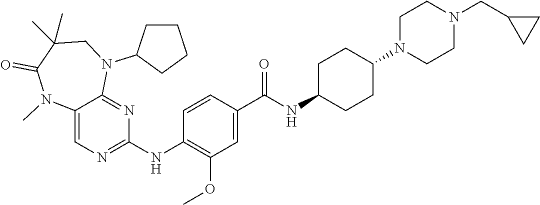 [SMILES: COc1cc(C(=O)N[C@H]2CC[C@H](N3CCN(CC4CC4)CC3)CC2)ccc1Nc1ncc2c(n1)N(C1CCCC1)CC(C)(C)C(=O)N2C] |
| A3 | 4-(9′-cyclopentyl-5′-methyl-6′-oxo-5′,6′,8′,9′- tetrahydrospiro[cyclobutane-1,7′-pyrimido[4,5- b][1,4]diazepine]-2′-ylamino)-N-((trans)-4-(4- (cyclopropylmethyl)piperazin-1-yl)cyclohexyl)- 3-methoxybenzamide | 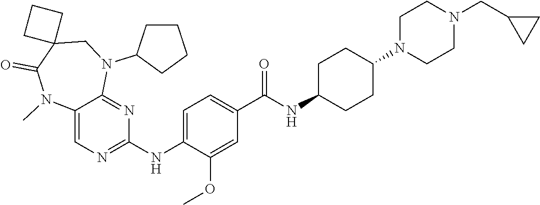 [SMILES: COc1cc(C(=O)N[C@H]2CC[C@H](N3CCN(CC4CC4)CC3)CC2)ccc1Nc1ncc2c(n1)N(C1CCCC1)CC1(CCC1)C(=O)N2C] |
| A4 | 4-(9-cyclopentyl-5-methyl-6-oxo-6,7,8,9- tetrahydro-5H-pyrimido[4,5-b][1,4]diazepin-2- ylamino)-N-((trans)-4-(4- (cyclopropylmethyl)piperazin-1-yl)cyclohexyl)- 3-methoxybenzamide | 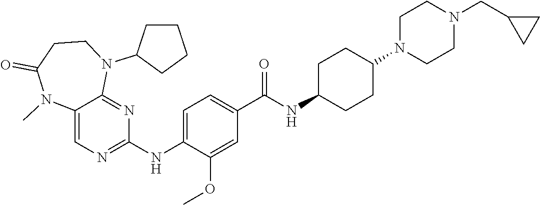 |
| A5 | 4-(9′-cyclopentyl-5′-methyl-6′-oxo-5′,6′,8′,9′- tetrahydrospiro[cyclopropane-1,7′- pyrimido[4,5-b][1,4]diazepine]-2′-ylamino)-N- ((trans)-4-(4-ethylpiperazin-1-yl)cyclohexyl)-3- methoxybenzamide | 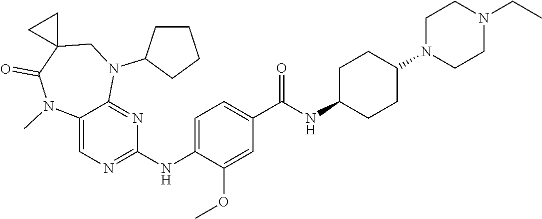 |
| A6 | 4-(9′-cyclopentyl-5′-methyl-6′-oxo-5′,6′,8′,9′- tetrahydrospiro[cyclopropane-1,7′- pyrimido[4,5-b][1,4]diazepine]-2′-ylamino)-N- ((cis)-4-(4-ethylpiperazin-1-yl)cyclohexyl)-3- methoxybenzamide | 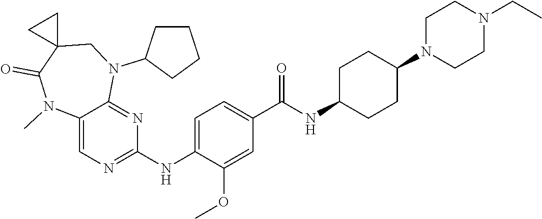 [SMILES: CCN1CCN([C@H]2CC[C@@H](NC(=O)c3ccc(Nc4ncc5c(n4)N(C4CCCC4)CC4(CC4)C(=O)N5C)c(OC)c3)CC2)CC1] |
| A7 | 4-(9′-cyclopentyl-5′-methyl-6′-oxo-5′,6′,8′,9′- tetrahydrospiro[cyclopropane-1,7′- pyrimido[4,5-b][1,4]diazepine]-2′-ylamino)-N- ((trans)-4-(4-methylpiperazin-1-yl)cyclohexyl)- 3-methoxybenzamide | 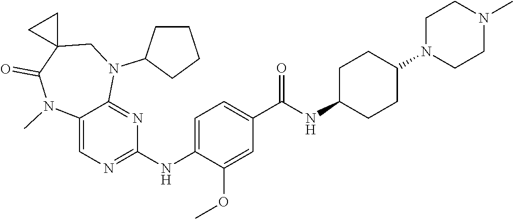 |
| A8 | 4-(9′-cyclopentyl-5′-methyl-6′-oxo-5′,6′,8′,9′- tetrahydrospiro[cyclopropane-1,7′- pyrimido[4,5-b][1,4]diazepine]-2′-ylamino)-N- ((cis)-4-(4-methylpiperazin-1-yl)cyclohexyl)-3- methoxybenzamide | 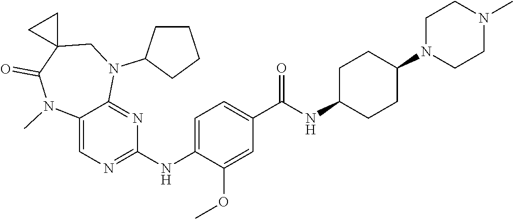 [SMILES: COc1cc(C(=O)N[C@H]2CC[C@@H](N3CCN(C)CC3)CC2)ccc1Nc1ncc2c(n1)N(C1CCCC1)CC1(CC1)C(=O)N2C] |
| A9 | N-((trans)-4-(4-benzylpiperazin-1- yl)cyclohexyl)-4-(9′-cyclopentyl-5′-methyl-6′- oxo-5′,6′,8′,9′-tetrahydrospiro[cyclopropane- 1,7′-pyrimido[4,5-b][1,4]diazepine]-2′-ylamino)- 3-methoxybenzamide | 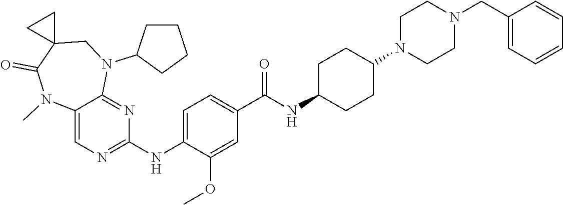 |
| A10 | N-((cis)-4-(4-benzylpiperazin-1-yl)cyclohexyl)- 4-(9′-cyclopentyl-5′-methyl-6′-oxo-5′,6′,8′,9′- tetrahydrospiro[cyclopropane-1,7′- pyrimido[4,5-b][1,4]diazepine]-2′-ylamino)-3- methoxybenzamide | 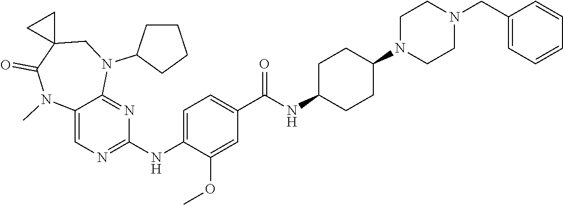 [SMILES: COc1cc(C(=O)N[C@H]2CC[C@@H](N3CCN(Cc4ccccc4)CC3)CC2)ccc1Nc1ncc2c(n1)N(C1CCCC1)CC1(CC1)C(=O)N2C] |
| A11 | 4-(9-cyclopentyl-5,7,7-trimethyl-6-oxo-6,7,8,9- tetrahydro-5H-pyrimido[4,5-b][1,4]diazepin-2- ylamino)-3-methoxy-N-((trans)-4- morpholinocyclohexyl)benzamide | 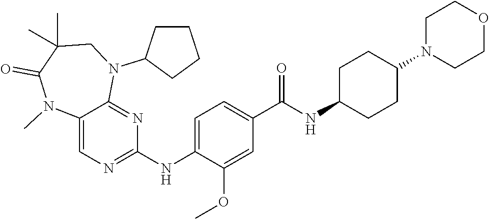 |
| A12 | 4-(9-cyclopentyl-5,7,7-trimethyl-6-oxo-6,7,8,9- tetrahydro-5H-pyrimido[4,5-b][1,4]diazepin-2- ylamino)-3-methoxy-N-((cis)-4- morpholinocyclohexyl)benzamide | 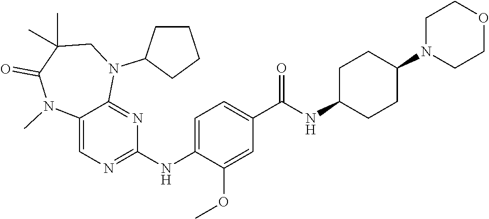 [SMILES: COc1cc(C(=O)N[C@H]2CC[C@@H](N3CCOCC3)CC2)ccc1Nc1ncc2c(n1)N(C1CCCC1)CC(C)(C)C(=O)N2C] |
| A13 | 4-(9-cyclopentyl-5-methyl-6-oxo-6,7,8,9- tetrahydro-5H-pyrimido[4,5-b][1,4]diazepin-2- ylamino)-3-methoxy-N-[1-(tetrahydro-pyran-4- yl)-piperidin-4-yl]-benzamide | 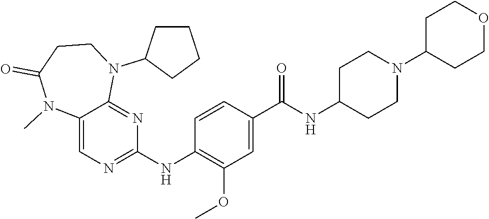 [SMILES: COc1cc(C(=O)NC2CCN(C3CCOCC3)CC2)ccc1Nc1ncc2c(n1)N(C1CCCC1)CCC(=O)N2C] |
| A14 | 4-(9′-cyclopentyl-5′-methyl-6′-oxo-5′,6′,8′,9′- tetrahydrospiro[cyclopropane-1,7′- pyrimido[4,5-b][1,4]diazepine]-2′-ylamino)-3- methoxy-N-((trans)-4- morpholinocyclohexyl)benzamide | 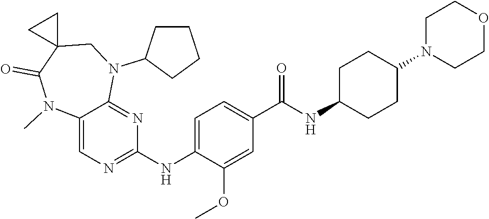 |
| A15 | 4-(9′-cyclopentyl-5′-methyl-6′-oxo-5′,6′,8′,9′- tetrahydrospiro[cyclopropane-1,7′- pyrimido[4,5-b][1,4]diazepine]-2′-ylamino)-3- methoxy-N-((cis)-4- morpholinocyclohexyl)benzamide | 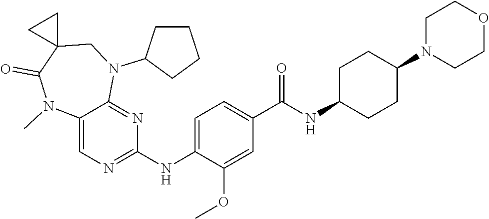 [SMILES: COc1cc(C(=O)N[C@H]2CC[C@@H](N3CCOCC3)CC2)ccc1Nc1ncc2c(n1)N(C1CCCC1)CC1(CC1)C(=O)N2C] |

### Cell Lines

Tumour sensitivity towards Plk1 inhibitors was studied in a panel of oesophageal (OE) cell lines. The cell lines used were: a benign Barrett's (CPA), two Barrett's adenocarcinoma (OE19 and OE33; Sigma Aldrich), and two oesophageal squamous cell carcinoma cell lines (OE21, ECACC; and KYSE-410, ECACC). The p53 mutational and functional status are shown in the table below.

TABLE 1
p53 mutational and functional status of OE cell lines

| Characteristic | CPA | KYSE-410 | OE21 | OE33 | OE19 |
|---|---|---|---|---|---|
| Type | Benign | ESCC | ESCC | BAC | BAC |
| | Barrett's | | | | |
| TP53 | WT | c.1009C > T | c.269C > T | c.404G > A | c.929dupA |
| mutation | | | c.270delC | | |
| p53 amino | Nil | R337C | S90fs31X | C135Y | N310K |
| acid change | | | | | Frame |
| p53 function | Yes | Yes | No | No | Yes |

Additionally the sensitivity towards Plk1 inhibitors was studied in lung (NCI-H1944 and NCI-H2122. ATCC), colorectal (HCT116 and Colo205. ECACC), breast (MCF7, ECACC, MCF10A, ATCC and MDA-MB231. ECACC), prostate (PC3, ECACC and DU-145, ATCC), leukaemia (HEL, DSMZ and HL-60. ECACC).

### Anti-Proliferative Effect of Plk1 Inhibitors

The anti-proliferative effect of Plk1 inhibitors was studied in a 96-well format drug washout/outgrowth assay. Cells were seeded at 1.5-2.5×10³ cells per well in 96-well plates and left to incubate overnight at 37° C., 5% CO₂, before addition of a range of concentrations of each of the different Plk1 inhibitors. Cells were incubated with the compounds for various periods of time, ranging from 3 to 24 hours; media containing the compounds was then removed from the cells and replaced with drug-free media until the end of the assay. The cell viability was evaluated on day 6 using a standard resazurin-based assay according to manufacturer's instructions (AlamarBlue, AbD Serotec, Oxford, UK). The IC50 values were determined using IDBS software.

### Measurement of Plasma Concentrations of Drug

The plasma concentration is measured using standard LC-MS/MS analysis based on the following method. Calibration and control samples are prepared as follows from a 500 μg/mL solution of compound in methanol/DMSA 50/50 v/v. This stock solution is used to prepare diluted working standards of the test items with concentrations from 200 ng/ml to 500,000 ng/ml. The solutions are diluted with methanol and stored at +4° C. in glass vials when not in use. The working solutions are used to prepare calibration standards in control plasma, with a final concentration of between 10 ng/ml and 5000 ng/ml per 100 microlitres of plasma. These standards are prepared on day one of analysis and re-prepared as necessary on each analytical occasion. Internal standards are dissolved in methanol/DMSA 50/50 v/v and diluted to 1 μg/mL in acetonitrile for a working solution.

Plasma samples are extracted by the following method. Transfer 50 microlitres of each calibration, quality control and test sample to a 96-well mictrotitre plate. Add 200 microlitres of a 1 microgram per millilitre internal standard solution in acetonitrile to all samples except for a double blank. 200 microlitres of acetonitrile is added to the double blank. Samples are briefly vortex mixed, and centrifuged at 2128 g for 30 minutes. Recoverable supernatant is transferred to an appropriate well of a microtitre plate and extracts are evaporated with oxygen free nitrogen at a gas temperature of about 40° C. Extracts are reconstituted in 200 μL of mobile phase and mixed well. The plate is sealed with silicone-based pre-pierced web seal and used for LC-MS/MS analysis.

### LC-MS/MS Analysis

LC-MS/MS analysis can be performed using the following chromatography conditions.

| | |
|---|---|
| Analytical Column: | XBridge C₁₈ 50 × 3 mm; 2.5 μm (Waters |
| | Corporation) |
| Column Temperature: | Ambient Temperature |
| Connecting tubing material: | PEEK |
| Mobile Phase: | 10 mM Ammonium Acetate |
| | pH10,/Methanol (35/65 v/v) |
| Flow rate: | 0.3 ml/min |
| Injection volume: | 10 μl |
| Loop size: | 20 μl |
| Autosampler wash: | Methanol/isoproponal/ |
| | acetone/TFA (40/30/30/0.5 v/v/v/v) |
| Rack Temperature: | +6° C. |

### Tandem Mass Spectrometry

| | |
|---|---|
| Interface: | Z-Spray interface with electrospray probe |
| Mode: | MRM (Multiple Reaction Monitoring) |
| Polarity: | Positive |
| Run Time: | 5 min |
| Dwell Time: | 0.08 s/transition |
| Q1 and Q3 resolution: | Unit |
| Capillary voltage: | 3.5 kV |
| Desolvation Temperature: | 450° C. |
| Desolvation Gas Flow (L/hr): | ca. 600 L/hr |
| Source Temperature: | 140° C. |
| Cone Gas Flow (L/hr): | ca. 60 L/hr |

### Reagents, Chemicals and Solvents

| | |
|---|---|
| Ammonium acetate | Certified grade |
| Acetonitrile | HPLC grade |
| Dimethyl suphoxide | Certified grade |
| Water | Milli-Q+ deionised |

### Analytical Instrumentation

| | |
|---|---|
| Autosampler: | Model 2777 (Waters/Micromass Corporation) |
| Degasser: | Model AF (Waters/Micromass Corporation) |
| HPLC Pump: | Model 1525 (Waters/Micromass Corporation) |
| Mass Spectrometer: | Quattro Premier (Waters/Micromass |
| | Corporation) |

### Disposable Supplies

| | |
|---|---|
| Pipettes | Microman (Gilson) |
| | 12-channel (Rainin) |
| Analytical Balance | AT261 (Mettler Toledo) |
| Centrifuge | Micro Centrifuge 2-16 (Sigma) |
| Vortex Mixer | Clifton Cyclone (Clifton) |
| Water Purifier | MilliQ+ (Waters) |

Alternative reagents, instrumentation, laboratory equipment and disposable supplies of equivalent specification may be used.

### Reagent Preparation

### Mobile Phase and Reconstitution Solvent

Transfer 650 ml of HPLC-grade methanol to a 1000 ml Duran bottle and add 350 ml of 10 mM ammonium acetate pH 10 and mix well. Sonicate for 10 minutes prior to use.

### Methanol/Water/Formic Acid 50/50/0.1 (Auto-Injector Wash)

Transfer 500 ml of HPLC grade methanol to 500 ml of MilliQ+ deionised water and add 1 mL of formic acid. Mix well. Sonicate for 10 minutes prior to use.

### Results

Pyrimidodiazepinone compounds inhibited the proliferation of the BAC and ESCC cell lines more potently than the benign OE cell line. In particular, compounds A1, A7 and A13 inhibited the proliferation of the BAC and ESCC cell lines at least 10-fold more potently than the benign OE cell line. 6 hour treatment provided the best differentiation between cancerous and non-cancerous cells lines. Compound BI2536 showed little or no differentiation between the cancerous and the benign cell lines at all timepoints. With the pyrimidodiazepinone compounds, the highest therapeutic window for cancerous over non-cancerous cell lines was observed for the OE cancer cell lines with mutant and non-functional p53.

Clinical Plk1 inhibitors investigated to date have demonstrated very small therapeutic windows. Most commonly, dose limiting toxicities and adverse events for these agents are grade 3/4 neutropenia and febrile neutropenia, thrombocytopenia and anemia, which are related to the inhibition of rapidly proliferating blood cells. To achieve a better therapeutic window a highly controlled, short term treatment to allow differentiation between sensitive cancer cells and normal proliferating cells is required. Long elimination half-life of Plk1 inhibitors will be disadvantageous. Competitive clinical molecules have relatively long half-life: GSK461364 9-13 h (Olmos et al. 2011), BI2536>25 h (Gandhi et al., 2009), BI6727˜110 h (Gil et al., 2010), MK1496 25-47h (Doi et al., 2011). Based on the mouse PK data (Table 2) pyrimidodiazepinone Plk1 inhibitors are expected to have significantly shorter half-life in humans, which will allow the required short term treatment.

TABLE 2
Mouse PK data for selected compounds of the invention, together with
prior art compound BI6727 (Rudolph et al.)

| | | Compound | Compound | Compound | |
|---|---|---|---|---|---|
| Parameter | Units | A13 | A1 | A7 | BI6727* |
| Dose | mg/kg | 1 | 1 | 1 | 35 |
| HL_Lambda_z | hr | 0.78 | 2.89 | 1.97 | 46 |
| Cmax | ng/ml | 405 | 478 | 453 | 4390 |
| AUCall | hr * ng/ml | 164 | 631 | 377 | |
| Vz_obs | ml/kg | 6713 | 5304 | 6937 | 7600 |
| Cl_obs | ml/hr/kg | 5988 | 1273 | 2445 | 600 |

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

### REFERENCES

Petronczki et al., Curr Opin Cell Biol 2008 December; 20(6):650-60;
Petronczki et al., Dev. Cell 2008 May; 14(5):646-59;
Kanaji et al., Oncology, 2006; 70(2):126-33;
Spankuch et al., Oncogene, 2007 Aug. 23; 26(39):5793-807;
Schoffski et al., Eur J Cancer, 2012, January; 48(2); 179-86;
Degenhardt et al, Clin Exp Metastasis. 2011 December; 28(8):899-908;
Olmos D et al., Phase I study of GSK461364, a specific and competitive polo-like kinese 1 inhibitor in patients with advanced solid tumors. 2011, Clin Cancer Res, 17, 3420;
Gandhi L et al. An open label phase II trial of the Plk1 inhibitor BI2536 in patients with sensitive relapse small cell lung cancerl; 2009 ASCO Annual Meeting, Abstract 8108;
Gil T et al. Final analysis of a phase I single dose-escalation study of the novel polo-like kinase inhibitor BI6727 in patients with advanced solid tumors; 2010 ASCO Annual Meeting, Abstract 3061;
Doi T et al. A first-in-human phase I dose-escalation study of MK-1496, first-in-class orally available novel Plk1 inhibitor, in patients with advanced solid tumors; 2011 ASCO Annual Meeting. Abstracts 3012;
Rudolph D et al. BI6727, a polo-like kinase inhibitor with improved pharmacokinetic profile and broad antitumor activity; 2009, Clin Cancer Res, 15:3094.

1. A method of treating a proliferative disorder, said method comprising administering to a subject a therapeutically effective amount of a compound of formula (I), or a pharmaceutically acceptable salt thereof, 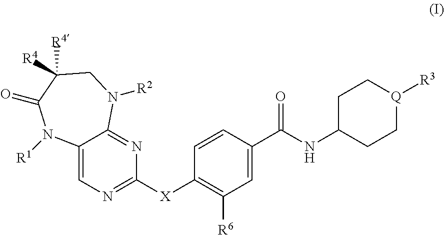
wherein:
X is NR⁷;
R¹ and R² are each independently H, alkyl or cycloalkyl;
R³ is a 6-membered heterocycloalkyl group selected from piperidinyl, piperazinyl, morpholinyl and tetrahydropyranyl, wherein said heterocycloalkyl group is optionally further substituted by one or more (CH₂)ₙR¹⁹ groups;
R⁴ and R^{4′} are each independently H or alkyl; or
R⁴ and R^{4′} together form a spiro cycloalkyl group;
Q is CH or N;
R⁶ is OR⁸ or halogen;
n is 1, 2 or 3;
R¹⁹ is H, alkyl, aryl or a cycloalkyl group;
R⁷ and R⁸ are each independently H or alkyl; and
wherein said compound is administered in accordance with a dosing regimen which:
(i) maintains a plasma concentration of from about 50 to about 500 nM for a period of up to about 16 hours; or
(ii) maintains a plasma concentration of from about 0.5 μM to about 1 μM for a period of up to about 6 hours; or
(iii) achieves a maximum plasma concentration (Cmax) of no more than about 500 nM within a period of about 6 hours; or
(iv) achieves a maximum plasma concentration (Cmax) of no more than about 200 nM within a period of about 16 hours; or
(v) achieves a maximum plasma concentration (Cmax) of about 0.5 μM to about 1 μM within about 6 hours.

2. The method of claim 1 wherein said compound is administered in a dosing regimen which maintains a plasma concentration of from about 100 to about 500 nM for a period of up to about 16 hours.

3. The method of claim 1 wherein said compound is administered in a dosing regimen which maintains a plasma concentration of from about 100 to about 500 nM for a period of up to about 6 hours.

4. The method of claim 1 wherein said compound is administered in a dosing regimen which maintains a plasma concentration of from about 100 to about 500 nM for a period of from about 3 to about 6 hours.

5. The method of claim 1 wherein said compound is administered in a dosing regimen which maintains a plasma concentration of from about 50 to about 250 nM for a period of up to about 16 hours.

6. The method of claim 5 wherein said compound is administered in a dosing regimen which maintains a plasma concentration of from about 50 to about 250 nM for a period of about 10 to about 16 hours.

7. The method of claim 1 wherein said compound is administered in a dosing regimen which maintains a plasma concentration of from about 0.5 μM to about 1 μM for a period of from about 3 to about 6 hours.

8. The method of claim 1 wherein said compound is administered in a dosing regimen which achieves a maximum plasma concentration (Cmax) of about 0.5 μM to about 1 μM within about 6 hours.

9. The method of claim 1 wherein said compound is administered in a dosing regimen which achieves a maximum plasma concentration (Cmax) of about 100 nM to about 500 nM within a period of about 6 hours.

10. The method of claim 1 wherein said compound is administered in a dosing regimen which achieves a maximum plasma concentration (Cmax) of about 50 nM to about 200 nM within a period of about 16 hours.

11. The method of claim 1 wherein the compound is administered by intravenous infusion.

12. The method of claim 1 wherein the compound is administered by intravenous infusion for about 1 to about 4 hours at a rate of about 0.04 to about 0.08 mg/kg/minute.

13. The method of claim 1 wherein the compound is administered by intravenous infusion for about 1 to about 2 hours at a rate of about 0.02 to about 0.06 mg/kg/minute.

14. The method of claim 1 wherein the compound is administered by intravenous infusion for about 1 to about 2 hours at a rate of about 0.02 to about 0.08 mg/kg/minute.

15. The method of claim 1 wherein the compound is administered by intravenous infusion for about 1 to about 10 hours at a rate of about 0.01 to about 0.04 mg/kg/minute.

16. The method of claim 1 which wherein the compound is administered by bolus injection.

17. The method of claim 16 wherein the compound is administered by two or more bolus injections.

18. The method of claim 1 wherein said compound is of formula (Ia), or a pharmaceutically acceptable salt thereof, 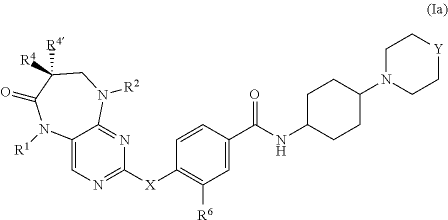
wherein Y is O or N—(CH₂)ₙR¹⁹; and
X, R¹, R², R⁴, R^{4′}, R⁶, R⁷, R⁸, n and R¹⁹ are as defined in claim 1.

19. The method of claim 1 wherein said compound is of formula (Ib), or a pharmaceutically acceptable salt thereof, 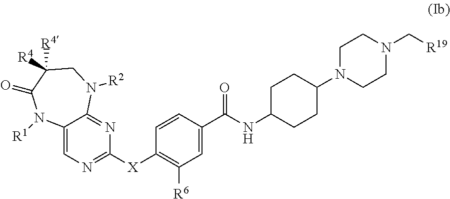
wherein X, R¹, R², R⁴, R^{4′}, R⁷, R⁸ and R¹⁹ are as defined in claim 1.

20. The method of claim 1, wherein said compound is selected from the following:

| | Name |
|---|---|
| A1 | 4-(9′-cyclopentyl-5′-methyl-6′-oxo-5′,6′,8′,9′-tetrahydrospiro[cyclopropane-1,7′-pyrimido |
| | [4,5-b][1,4]diazepine]-2′-ylamino)-N-((trans)-4-(4-(cyclopropylmethyl)piperazin-1-yl) |
| | cyclohexyl)-3-methoxybenzamide |
| A2 | 4-(9-cyclopentyl-5,7,7-trimethyl-6-oxo-6,7,8,9-tetrahydro-5H-pyrimido[4,5-b] [1,4] |
| | diazepin-2-ylamino)-N-((trans)-4-(4-(cyclopropylmethyl)piperazin-1-yl)cyclohexyl)-3- |
| | methoxybenzamide |
| A3 | 4-(9′-cyclopentyl-5′-methyl-6′-oxo-5′,6′,8′,9′-tetrahydrospiro[cyclobutane-1,7′-pyrimido |
| | [4,5-b][1,4]diazepine]-2′-ylamino)-N-((trans)-4-(4-(cyclopropylmethyl)piperazin-1- |
| | yl)cyclohexyl)-3-methoxybenzamide |
| A4 | 4-(9-cyclopentyl-5-methyl-6-oxo-6,7,8,9-tetrahydro-5H-pyrimido[4,5-b][1,4]diazepin-2- |
| | ylamino)-N-((trans)-4-(4-(cyclopropylmethyl)piperazin-1-yl)cyclohexyl)-3- |
| | methoxybenzamide |
| A5 | 4-(9′-cyclopentyl-5′-methyl-6′-oxo-5′,6′,8′,9′-tetrahydrospiro[cyclopropane-1,7′-pyrimido |
| | [4,5-b][1,4]diazepine]-2′-ylamino)-N-((trans)-4-(4-ethylpiperazin-1-yl)cyclohexyl)-3- |
| | methoxybenzamide |
| A6 | 4-(9′-cyclopentyl-5′-methyl-6′-oxo-5′,6′,8′,9′-tetrahydrospiro[cyclopropane-1,7′-pyrimido |
| | [4,5-b][1,4]diazepine]-2′-ylamino)-N-((cis)-4-(4-ethylpiperazin-1-yl)cyclohexyl)-3- |
| | methoxybenzamide |
| A7 | 4-(9′-cyclopentyl-5′-methyl-6′-oxo-5′,6′,8′,9′-tetrahydrospiro[cyclopropane-1,7′-pyrimido |
| | [4,5-b][1,4]diazepine]-2′-ylamino)-N-((trans)-4-(4-methylpiperazin-1-yl)cyclohexyl)-3- |
| | methoxybenzamide |
| A8 | 4-(9′-cyclopentyl-5′-methyl-6′-oxo-5′,6′,8′,9′-tetrahydrospiro[cyclopropane-1,7′-pyrimido |
| | [4,5-b][1,4]diazepine]-2′-ylamino)-N-((cis)-4-(4-methylpiperazin-1-yl)cyclohexyl)-3- |
| | methoxybenzamide |
| A9 | N-((trans)-4-(4-benzylpiperazin-1-yl)cyclohexyl)-4-(9′-cyclopentyl-5′-methyl-6′-oxo- |
| | 5′,6′,8′,9′-tetrahydrospiro[cyclopropane-1,7′-pyrimido[4,5-b][1,4]diazepine]-2′-ylamino)- |
| | 3-methoxybenzamide |
| A10 | N-((cis)-4-(4-benzylpiperazin-1-yl)cyclohexyl)-4-(9′-cyclopentyl-5′-methyl-6′-oxo- |
| | 5′,6′,8′,9′-tetrahydrospiro[cyclopropane-1,7′-pyrimido[4,5-b][1,4]diazepine]-2′-ylamino)- |
| | 3-methoxybenzamide |
| A11 | 4-(9-cyclopentyl-5,7,7-trimethyl-6-oxo-6,7,8,9-tetrahydro-5H-pyrimido[4,5-b][1,4] |
| | diazepin-2-ylamino)-3-methoxy-N-((trans)-4-morpholinocyclohexyl)benzamide |
| A12 | 4-(9-cyclopentyl-5,7,7-trimethyl-6-oxo-6,7,8,9-tetrahydro-5H-pyrimido[4,5-b][1,4] |
| | diazepin-2-ylamino)-3-methoxy-N-((cis)-4-morpholinocyclohexyl)benzamide |
| A13 | 4-(9-cyclopentyl-5-methyl-6-oxo-6,7,8,9-tetrahydro-5H-pyrimido[4,5-b][1,4]diazepin-2- |
| | ylamino)-3-methoxy-N-[1-(tetrahydro-pyran-4-yl)-piperidin-4-yl]-benzamide |
| A14 | 4-(9′-cyclopentyl-5′-methyl-6′-oxo-5′,6′,8′,9′-tetrahydrospiro[cyclopropane-1,7′-pyrimido |
| | [4,5-b][1,4]diazepine]-2′-ylamino)-3-methoxy-N-((trans)-4-morpholinocyclohexyl) |
| | benzamide |
| A15 | 4-(9′-cyclopentyl-5′-methyl-6′-oxo-5′,6′,8′,9′-tetrahydrospiro[cyclopropane-1,7′- |
| | pyrimido[4,5-b][1,4]diazepine]-2′-ylamino)-3-methoxy-N-((cis)-4- |
| | morpholinocyclohexyl)benzamide |

and pharmaceutically acceptable salts thereof.

21. The method of claim 1 wherein the proliferative disorder is cancer or leukemia.

22. The method of claim 21 wherein the cancer is a solid tumour.

23. The method of claim 21 wherein the cancer comprises a non-functional p53 protein.

24. The method of claim 21 wherein the cancer comprises a p53-mutation.

25. The method of claim 21 wherein the cancer is selected from breast cancer, colorectal cancer, prostate cancer, oesophageal cancer and lung cancer.

26. The method of claim 1 wherein said compound is administered in combination with at least one pharmaceutically acceptable diluent, excipient or carrier.

27-28. (canceled)

29. A kit comprising:
(a) a compound of formula (I) according to claim 1, or a pharmaceutically acceptable salt thereof, 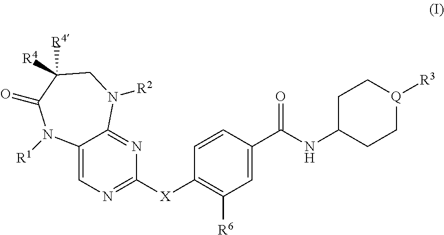
wherein:
X is NR⁷;
R¹ and R² are each independently H, alkyl or cycloalkyl;
R³ is a 6-membered heterocycloalkyl group selected from piperidinyl, piperazinyl, morpholinyl and tetrahydropyranyl, wherein said heterocycloalkyl group is optionally further substituted by one or more (CH₂)ₙR¹⁹ groups;
R⁴ and R^{4′} are each independently H or alkyl; or
R⁴ and R^{4′} together form a spiro cycloalkyl group;
Q is CH or N;
R⁶ is OR⁸ or halogen;
n is 1, 2 or 3;
R¹⁹ is H, alkyl, aryl or a cycloalkyl group;
R⁷ and R⁸ are each independently H or alkyl;
(b) at least one pharmaceutically acceptable diluent, excipient or carrier; and
(c) instructions to administer the compound of formula (I) in accordance with a dosing regimen which:
(i) maintains a plasma concentration of from about 50 to about 500 nM for a period of up to about 16 hours; or
(ii) maintains a plasma concentration of from about 0.5 μM to about 1 μM for a period of up to about 6 hours; or
(iii) achieves a maximum plasma concentration (Cmax) of no more than about 500 nM within a period of about 6 hours; or
(iv) achieves a maximum plasma concentration (Cmax) of no more than about 200 nM within a period of about 16 hours; or
(v) achieves a maximum plasma concentration (Cmax) of about 0.5 μM to about 1 μM within about 6 hours.

30. A method of treating a proliferative disorder, said method comprising administering to a subject a therapeutically effective amount of a compound of formula (I), or a pharmaceutically acceptable salt thereof, 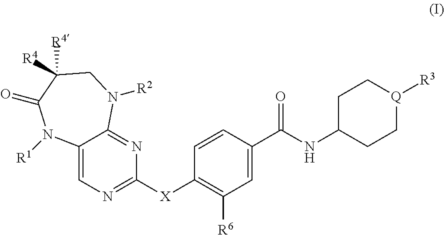
wherein:
X is NR⁷;
R¹ and R² are each independently H, alkyl or cycloalkyl;
R³ is a 6-membered heterocycloalkyl group selected from piperidinyl, piperazinyl, morpholinyl and tetrahydropyranyl, wherein said heterocycloalkyl group is optionally further substituted by one or more (CH₂)ₙR¹⁹ groups;
R⁴ and R^{4′} are each independently H or alkyl; or
R⁴ and R^{4′} together form a spiro cycloalkyl group;
Q is CH or N;
R⁶ is OR⁸ or halogen;
n is 1, 2 or 3;
R¹⁹ is H, alkyl, aryl or a cycloalkyl group;
R⁷ and R⁸ are each independently H or alkyl; and
wherein said compound is administered by:
intravenous infusion for about 1 to about 4 hours at a rate of about 0.02 to about 0.08 mg/kg/minute; or
intravenous infusion for about 1 to about 10 hours at a rate of about 0.01 to about 0.04 mg/kg/minute.

31. The method of claim 30 wherein the compound is administered by intravenous infusion for about 1 to about 4 hours at a rate of about 0.04 to about 0.08 mg/kg/minute.

32. The method of claim 30 wherein the compound is administered by intravenous infusion for about 1 to about 2 hours at a rate of about 0.02 to about 0.06 mg/kg/minute.

33. The method of claim 30 wherein the compound is administered by intravenous infusion for about 1 to about 3 hours at a rate of about 0.02 to 0.08 mg/kg/minute.
